SEQUENCE LISTING (9 sequences)

SEQ ID NO: 1 - DNA, 2060 bp, Candida albicans
ctttattaggaagattggctaggccattttgtattacgggtctccaaagtgcaattgttttagtaaatatccaatcattgggcttcagtgtgaatgggggttgtcaatctcttggtgtagaaataggcgcaggcctccgaatcccaaaaaaagaagaatcaggatgtctcggctgcaagatttgtagccatggcaaatgccgaaaaatgaaaaaaaaaaaaaagtctactgggcccacctacaaaaggaaaagtgattgaactagatcagtagtggtctggaccctctataattttataatattgtcacgggctttagaatttgtataattgtgtgtctgacactctgtggttaatatctggacatctcgttccccttgtgaagggtcgtctgtaatgaattcatgatcaagaataatatgactttgctcacttcatagagtgccgacttgattattattgagctttatcctctgtaatatatcgtaaccacttgacttatttccttgttgtgggattcactttggatgatgatgttaaccaaatgtaattggtacaatcctttttgtccttgtcgcgacttcctttaatatcgcgacttatttcattaatgagacgcaacgcattcctctctccatagaaaaaaaaaataacaaactgaaaaaataaacagcggacctcatctctttttttcaaatccactttttattactttattcaatgagtgaaagtgacgaaaccaaatcgatatcatctttaatatcttcttcttcttcatcacgtcccaaaaagtatatttgcacatatgaagggtgtgataaagcctataatcgaccatcattattagagcaacatttaagaacccacagtaatgatcgaccgtataaatgtacagtggacgattgtgataaagcatttttcagaaaatcacatttggaaacacatattgtatcacattccgaaaaaaaaccattccattgttcagtgtgtggtaaaggggttaattctcgacaacacttgaaaagacatgaaatcacccatacaaagtcatttaaatgtacatttgaaaattgtcaagaagcattttataaacatcaatctttaagacatcatatattatctgttcatgaaaaaacattaacgtgtaaacaatgtaataaagttttcactcgaccttcaaaattagcacaacataaattaaaacatcatggtggatctcctgcttatcaatgtgatcatcctggttgttttaaaaatttccaaacttggtcagtattacaatttcatataaaacaactgcatccaaaacttaaatgtcctaaatgtggtaaaggttgtgttgggaaaaaaggtttatcttcacatatgttaagtcatgatgattctaccatgatcaaaatatggacttgtgattattgtgatgtggggaaatttgcaaagaaaaatgaattagttgaacattataatatcttccatgatggtaatatccctgatgatttattaaaggaaactgaagtgaaaaaattagagaacctattagatcaaggatcgaaattaaataatttgcatgaattagaaacagagaaattaaaagtggaagaagatgaagaagatgaagaagatagtctagatgaaaaaagaagtgatgttagatcagactcaatgtcagctcaaagatcaataaaatcatttactgcttctttggaaggttcaaagagtgtttctaaacttattctgaatagtgggaagaagatcaattgtcctaagaataattgtgatagaatgttttctagagaatatgatttacgtcgacatttgaaatggcatgatgataatttacaaagaattgagtcattcttaaatagtatagaaaaagaagaaactccagaaggtgaaccattggttaaaaaagccaggatggatttattgccaaatgaaacatcagtgatttctcgataatatacatttaaaattatattaacatttttatttcctttaatttttattttttgtgggctttttattttacattatttaacttgacatattactctcttaatg

SEQ ID NO: 2 - DNA, 1239 bp, Candida albicans
  CDS (1)..(1236)
atgagtgaaagtgacgaaaccaaatcgatatcatctttaatatcttcttcttcttcatcacgtcccaaaaagtatatttgcacatatgaagggtgtgataaagcctataatcgaccatcattattagagcaacatttaagaacccacagtaatgatcgaccgtataaatgtacagtggacgattgtgataaagcatttttcagaaaatcacatttggaaacacatattgtatcacattccgaaaaaaaaccattccattgttcagtgtgtggtaaaggggttaattctcgacaacacttgaaaagacatgaaatcacccatacaaagtcatttaaatgtacatttgaaaattgtcaagaagcattttataaacatcaatctttaagacatcatatattatctgttcatgaaaaaacattaacgtgtaaacaatgtaataaagttttcactcgaccttcaaaattagcacaacataaattaaaacatcatggtggatctcctgcttatcaatgtgatcatcctggttgttttaaaaatttccaaacttggtcagtattacaatttcatataaaacaactgcatccaaaacttaaatgtcctaaatgtggtaaaggttgtgttgggaaaaaaggtttatcttcacatatgttaagtcatgatgattctaccatgatcaaaatatggacttgtgattattgtgatgtggggaaatttgcaaagaaaaatgaattagttgaacattataatatcttccatgatggtaatatccctgatgatttattaaaggaaactgaagtgaaaaaattagagaacctattagatcaaggatcgaaattaaataatttgcatgaattagaaacagagaaattaaaagtggaagaagatgaagaagatgaagaagatagtctagatgaaaaaagaagtgatgttagatcagactcaatgtcagctcaaagatcaataaaatcatttactgcttctttggaaggttcaaagagtgtttctaaacttattctgaatagtgggaagaagatcaattgtcctaagaataattgtgatagaatgttttctagagaatatgatttacgtcgacatttgaaatggcatgatgataatttacaaagaattgagtcattcttaaatagtatagaaaaagaagaaactccagaaggtgaaccattggttaaaaaagccaggatggatttattgccaaatgaaacatcagtgatttctcgataa

SEQ ID NO: 3 - protein, 412 aa, Candida albicans
MSESDETKSISSLISSSSSSRPKKYICTYEGCDKAYNRPSLLEQHLRTHSNDRPYKCTVDDCDKAFFRKSHLETHIVSHSEKKPFHCSVCGKGVNSRQHLKRHEITHTKSFKCTFENCQEAFYKHQSLRHHILSVHEKTLTCKQCNKVFTRPSKLAQHKLKHHGGSPAYQCDHPGCFKNFQTWSVLQFHIKQSHPKLKCPKCGKGCVGKKGLSSHMLSHDDSTMIKIWTCDYCDVGKFAKKNELVEHYNIFHDGNIPDDLLKETEVKKLENLLDQGSKLNNLHELETEKLKVEEDEEDEEDSLDEKRSDVRSDSMSAQRSIKSFTASLEGSKSVSKLISNSGKKINCPKNNCDRMFSREYDLRRHLKWHDDNLQRIESFLNSIEKEETPEGEPLVKKARMDLLPNETSVISR

SEQ ID NO: 4 - DNA, 21 bp, Candida albicans
atgagtgaaagtgacgaaacc

SEQ ID NO: 5 - DNA, 24 bp, Candida albicans
attggaatggttttttttcggaat

SEQ ID NO: 6 - DNA, 23 bp, Candida albicans
tggtttcgtcactttcactcatt

SEQ ID NO: 7 - DNA, 25 bp, Candida albicans
atgttaagtcatgatgattctacca

SEQ ID NO: 8 - DNA, 27 bp, Candida albicans
ccttagaattcaccatgagtgaaagtg

SEQ ID NO: 9 - DNA, 27 bp, Candida albicans
gctgagctcgagtattatcgagaaatc

(19) United States
(12) Patent Application Publication
(10) Pub. No.: US 2005/0158778 A1
(43) Pub. Date: Jul. 21, 2005

(54) Candida albicans tfIIIA gene (CATFIIIA) and the coded CATFIIIA protein

(75) Inventors: Florence Bordon-Pallier, Guyancourt (FR); Sylvie Camier, Piemont, CA (US); Andre Sentenac, Gif sur Yvette (FR)

Correspondence Address: MUSERLIAN, LUCAS AND MERCANTI, LLP, 475 PARK AVENUE SOUTH, 15TH FLOOR, NEW YORK, NY, 10016, US

(73) Assignee: Aventis Pharma S.A.

(21) Appl. No.: 11/026,156

(22) Filed: Dec. 30, 2004

Related U.S. Application Data
(62) Division of application No. 09/831,804, filed on Jul. 23, 2001.

(30) Foreign Application Priority Data
Nov. 10, 1998 (FR) 98/14147

(51) Int. Cl. C12Q 1/00; C12Q 1/68; C07H 21/04; C12N 1/18; C12N 15/74; C07K 14/40

(52) U.S. Cl. 435/6; 435/69.1; 435/254.22; 435/483; 530/350; 536/23.7

(57) ABSTRACT

A polypeptide having the transcription factor function CATFIIIA and having the amino acid sequence SEQ ID No: 3 coded by the DNA sequence of the CAtfIIIA gene coding for a protein having the biological function of transcription factor of candida albicans CATFIIIA containing the nucleotide sequence of SEQ ID No: 1 and the analogues of this peptide.

The present invention relates to the Candida albicans transcription factor hereafter called CATFIIIA and its analogues as well as the polynucleotides (RNA, DNA) coding for this protein or for the polypeptide analogues of this protein.

The present invention also relates to the preparation process for these polypeptides and polynucleotides, their use for the study of the transcription mechanisms in Candida albicans and for the preparation of inhibitors of this transcription factor CATFIIIA which can be used as an antifungal agent, and the pharmaceutical compositions containing such inhibitors.

Therefore the present invention in particular relates to a new transcription factor of Candida albicans and the DNA sequence coding for this transcription factor, their preparation and their uses.

We will also use hereafter the following abbreviations: AA for amino acids, NA for nucleic acids, RNA for ribonucleic acid, RNase for ribonuclease, DNA or DNA for deoxyribonucleic acid, cDNA for complementary DNA, bp for base pairs, PCR for polymerase chain reaction, CA or Candida a. for Candida albicans and SC or Saccharomyces c. for Saccharomyces cerevisiae.

The term screening which designates a specific screening technique and the term primer which designates an oligonucleotide used as a primer will also be used.

The term polynucleotide hereafter designates the polynucleotides of the present invention i.e. the DNA sequences and also the RNA sequences coding for the CATFIIIA factor of the present invention and its homologues having the same-transcription factor function. The term CAtfIII has the meaning given above for polynucleotides.

The term polypeptides designates hereafter the polypeptides of the present invention i.e. the CATFIIIA factor of the present invention and its functional analogues or homologues as defined hereafter, thus having the same transcription factor function. The term CATFIII has the meaning given to polypeptides above.

We will call the gene coding for the transcription factor TFIIIA tfIIIA (or tfC2) while CAtfIIIA (or CAtfC2) designates the gene coding for the transcription factor CATFIIIA of Candida albicans.

The range of known fungal infections extends from fungal attack of the skin or nails to more serious mycotic infections of internal organs. Such infections and the diseases which result from them, such as mycosis are identified. Antimycotic substances with fungistatic or fungicidal effects are used for the treatment of these mycoses.

The present invention thus relates to the identification of antimycotic substances and in particular anti-Candida albicans substances.

The present invention thus relates to inhibitors of transcription factors which can be used as antifungal agents. Candida albicans is a pathogenic yeast which causes infectious diseases in the human body. With the aim of finding of a means of treating diseases, intracellular targets can be chosen and the transcription factor TFIIIA can be one of these targets.

In eucaryotic organisms, this factor plays a key role in the initiation of transcription of 5S RNA genes by RNA-polymerase III. In particular for SC which is a similar yeast to CA, it has been shown that this SC yeast could not survive without an additional source of 5S RNA when the chromosomal gene of factor TFIIIA was interrupted, this additional 5S RNA being synthesized using a plasmid without the participation of factor TFIIIA (reference: S. Camier, A.-M. Dechampesme, A. Sentenac./Proc. Natl. Acad. Sci. (1995) 92, 9338-9342).

The tfIIInd gene and the corresponding TFIIIA protein are involved in regulation of the biological transcription mechanism as indicated below.

Since the TFIII protein was purified as transcription factor for the first time in 1980 from Xenopus ovocytes [Segall and al. Biol. Chem., 255, 11986-11991 (1980)], work has been carried out in vivo and in vitro in the Xenopus in order to study the transcription control mechanism exercized by TFIIIA. It has thus been shown that Xenopus TFIIIA is necessary for the initiation of transcription of the 5S RNA gene [Sakonji and al, Cell 19, 13-25 (1980)] and binds to an internal control region of the 5S RNA gene [Bogenhagen and al, Cell, 19,27-35 (1980)].

The nucleotide sequence of the cDNA of Xenopus TfIIIA and the corresponding amino acid sequence have already been published [Ginberg et al, Cell, 39.479-489 (1984)]. It can be noted that this gene codes for a protein having 9 zinc fingers, a zinc finger corresponding to a moiety containing two cysteines and two histidines linked by a zinc atom (CYS2 HIS2) (C2H2). This zinc finger structure constitutes a linking domain of proteins to the DNA and is therefore considered as an essential domain for a group of proteins which bind to DNA (DNA binding proteins).[Miller et al, Embo J., 4, 1607-1614 (1985)]

It can be noted that other transcription factors binding to DNA which also have this zinc finger structure are known such as for example, in human beings, XT1 of the Wilms human tumor gene, [Gessier et al, Nature, 343, 774-778 (1990)], the human transcription repressor YY1 [Shi et al, Cell, 67, 377-388 (1991)], the MAZ protein combined with the promoter cMYC [Bossone et al, Proc. Natl. Acad. Sci., USA, 89, 7452-7456 (1992)] or also sp1 [Kuwahara et al, J. Biol. Chem, 29, 8627-8631 (1990)].

The study of different organisms such as human beings in particular, the Xenopus or Candida albicans has shown that what can be called a family of TFIIIA transcription factors exist which have the following characteristics:
they are combined with RNA polymerase III
they have 9 zinc fingers
they are indispensable for the transcription of the gene coding for 5S RNA.

A known essential function of the protein coded by the tfIIIA gene (tfC2) in yeast is to initiate the transcription of the 5S RNA gene in Saccharomyces cerevisiae (Camier et al., Proc. Natl. Acad. Sa USA (1995) 92: 9338-9342).

The present invention has thus made it possible to isolate the DNA and RNA polynucleotides coding for the protein of the transcription factor CATFIIIA of Candida albicans and to reveal their nucleotide sequences.

A subject of the present invention is therefore an isolated polynucleotide containing a nucleotide sequence chosen from the following group:
a) a polynucleotide having at least 50% or at least 60% and preferably at least 70% identity with a polynucleotide coding for a polypeptide having the transcription factor function and having an amino acid sequence homologous with the sequence SEQ ID No 3 indicated hereafter.
b) a complementary polynucleotide of polynucleotide a)
c) a polynucleotide comprising at least 15 consecutive bases of the polynucleotide defined in a) and b).

A subject of the present invention is therefore a polynucleotide defined above in that this polynucleotide is a DNA.

A subject of the present invention is therefore a polynucleotide defined above in that this polynucleotide is an RNA.

A more precise subject of the present invention is the polynucleotide as defined above comprising the nucleotide sequence SEQ ID No 1.

The present invention has thus made it possible to isolate the DNA sequence coding for the transcription factor CATFIIIA of Candida albicans.

The present invention has also made it possible to reveal the nucleic acid sequence of the CAtfIIIA gene and also the amino acid sequence of the CATFIIIA protein coded by this gene.

A subject of the present invention is therefore a DNA sequence as defined by the polynucleotide above, characterized in that this DNA sequence is that of the CAtfIIIA gene coding for a protein having the biological function of transcription factor CATFIIIA of Candida albicans and containing the nucleotide sequence SEQ ID No1. Such a SEQ ID no1 sequence of the present invention therefore comprises 2060 nucleotides.

A precise subject of the present invention is a DNA sequence as defined above having the sequence starting at nucleotide 720 and finishing at nucleotide 1955 of SEQ ID No 1.

Such a sequence thus comprises 1236 nucleotides.

A subject of the present invention is also the DNA sequence of the CAtfIIIA gene as defined above coding for the amino acid sequence SEQ ID No 3.

The sequence SEQ ID No 3 thus comprises 412 AA.

A particular subject of the present invention is the DNA sequence coding for the transcription factor CATFIII as defined above as well as the DNA sequences which hybridize with it and/or have a significant homology with this sequence or of the fragments of it and having the same function.

A subject of the present invention is also a DNA sequence as defined above, comprising modifications introduced by suppression, insertion and/or substitution of at least one nucleotide coding for a protein with the same biological activity as the transcription factor CATFIIIA.

A particular subject of the present invention is the DNA sequence as defined above as well as DNA sequences which have a nucleotide sequence homology of at least 50% or at least 60% and preferably at least 70% with the said DNA sequence.

Therefore a subject of the present invention is also the DNA sequence as defined above as well as the DNA sequences which code for a protein of similar function, the AA sequence of which has a homology of at least 40% and in particular 45% or of at least 50%, rather at least 60% and preferably at least 70% with the AA sequence coded by the said DNA sequence.

By sequences which hybridize, are included the DNA sequences which hybridize with one of the DNA sequences above under standard conditions of high, medium or low stringency and which code for a polypeptide having the same transcription factor function. The stringency conditions are those carried out under the conditions known to a person skilled in the art such as those described by Sambrook et al, Molecular cloning, Cold Spring Harbor Laboratory Press, 1989. Such stringency conditions are for example hybridization at 65° C., for 18 hours in a 5×SSPE; 10×Denhardt's; 100 μg/ml ssDNA; 1% SDS solution followed by washing 3 times for 5 minutes with 2×SSC; 0.05% SDS, then washing 3 times for 15 minutes at 65° C. in 1×SSC; 0.1% SDS. High stringency conditions include for example hybridization at 65° C. for 18 hours in a 5×SSPE; 10× Denhardt; 100 μg/ml ssDNA; 1% SDS solution followed by washing twice for 20 minutes with a 2×SSC; 0.05% SDS solution at 65° C., followed by a final washing for 45 minutes in a 0.1×SSC; 0.1% SDS solution at 65° C. Medium stringency conditions include for example a final washing for 20 minutes in a 0.2×SSC, 0.1% SDS solution at 65° C.

By sequences which have a significant homology, are included sequences with a moderate or high nucleotide sequence similarity with one of the DNA sequences above and which code for a protein having the same transcription factor function.

By similar DNA sequence, is therefore meant DNA sequences which can belong to mycetes other than Candida albicans and in particular to SC, and which are similar or identical to the DNA sequence of the Candida albicans CatfIIIA gene. These similar DNA sequences are not necessarily identical to the DNA sequence of the Candida albicans CatfIIIA gene. The sequence homology at nucleotide level can be moderate or high. The present invention thus relates in particular to DNA sequences which have a nucleotide sequence homology of at least 50%, preferably at least 60% and even more preferably at least 70% with the CAtfIIIA sequence of the present invention.

In addition, these similar DNA sequences do not necessarily code for identical proteins, at the amino acid sequence level, to the protein coded by the CAtfIIIA gene. The present invention therefore relates in particular to DNA sequences which code for proteins said to be homologous, having an amino acid sequence homology of at least 40%, in particular 45%, preferably at least of 50%, more preferably at least of 60% and even more preferably at least of 70% with the protein coded by CAtfIIIA of the present invention.

The gene of the present invention is represented as a single strand DNA sequence as indicated in SEQ ID No 1 but it is understood that the present invention includes the complementary DNA sequence of this single strand DNA sequence and also includes the DNA sequence said to be double stranded constituted by these two DNA sequences complementary to each other.

The DNA sequence as defined above is an example of a combination of codons coding for the amino acids corresponding to the amino acid sequence SEQ ID No 3, but it is also understood that the present invention includes any other arbitrary combination of codons coding for this same amino acid sequence SEQ ID No 3.

For the preparation of polynucleotides and in particular DNA sequences as defined above, modified DNA sequences as indicated above or also homologous DNA sequences as defined above, techniques known to a person skilled in the art and in particular those described in the book by Sambrook, J. Fritsh, E. F. § Maniatis, T. (1989) entitled: ‘Molecular cloning: a laboratory manual, Laboratory, Cold Spring Harbor N.Y. can be used.

The homologous DNA sequences as defined above can in particular be isolated according to the methods known to a person skilled in the art for example by PCR technique using degenerated nucleotide primers to amplify these DNA from gene banks or cDNA banks of the corresponding mycetes. The cDNA can also be prepared from mRNA isolated from mycetes of different species studied within the scope of the present invention such as Candida albicans but also for example: Candida stellatoidea, Candida tropicalis, Candida parapsilosis, Candida krusei, Candida pseudotropicalis, Candida quillermondii, Candida glabrata, Candida lusianiae or Candida rugosa or also mycetes such as Saccharomyces cerevisiae or also Aspergillus or Cryptococcus and in particular, for example, Aspergillus fumigatus, Coccidioides immitis, Cryptococcus neoformans, Histoplasma capsulatum, Blastomyces dermatitidis, Paracoccidioides brasiliens and Sporothrix schenckii type mycetes or also mycetes of the classes of phycomycetes or eumycetes, in particular the sub-classes of basidiomycetes, ascomycetes, mehiascomycetales (yeast) and plectascales, gymnascales (skin and hair fungi) or of the hyphomycetes class, in particular the conidiosporales and thallosporales sub-classes amongst which are the following species: mucor, rhizopus, coccidioides, paracoccidioides (blastomyces, brasiliensis), endomyces (blastomyces), aspergillus, menicilium (scopulariopsis), trichophyton (ctenomyces), epidermophton, microsporon, piedraia, hormodendron, phialophora, sporotrichon, cryptococcus, candida, geotrichum, trichosporon or also toropsulosis.

The polynucleotides of the present invention can thus be obtained by using the usual cloning and screening methods such as those of cloning and sequencing from fragments of chromosomal DNA extracted from cells. For example, in order to obtain the polynucleotides of the present invention, a bank of chromosomal DNA fragments can be used. A probe corresponding to an oligonucleotide labelled with a radioactive element, preferably constituted by 17 or more nucleotides and derived from a partial sequence can be prepared. The clones containing DNA identical to that of the probe can be thus identified under stringent conditions. By the sequencing of the thus identified individual clones, using the sequencing primers originating from the original sequence, it is then possible to extend the sequence in both directions in order to determine the complete gene sequence. In a usual and efficient fashion, such sequencing can be carried out by using denatured double strand DNA prepared from a plasmid. Such techniques are described by Maniatis, T. Fritsch, E. F. and Sambrook as indicated above.(Laboratory Manual, Cold Spring Harbor, N.Y. (1989) (in particular in 1.90 and 13.70 in the chapters of screening by hybridization and sequencing from denatured double strand DNA).

Within the scope of the present invention, a bank of chromosomal DNA fragments of Candida albicans can in particular be used as indicated hereafter in Example 1 in the experimental part.

A detailed description of the operating conditions in which the present invention has been carried out is given below.

A very particular subject of the present invention is the polypeptide having the transcription factor function CATFIIIA and having the amino acid sequence SEQ ID No 3 coded by the DNA sequence as defined above and the analogues of this polypeptide.

By polypeptide analogues, are understood polypeptides, the amino acid sequence of which has been modified by substitution, suppression or addition of one or more amino acids but which retain the same biological function. Such polypeptide analogues can be produced spontaneously or can be produced by post-transcriptional modification or also by modification of the DNA sequence of the present invention as indicated above, using techniques known to a person skilled in the art: Amongst these techniques, the technique of directed mutagenesis known to a person skilled in the art (Kramer, W., et al., Nucl. Acids Res., 12, 9441 (1984); Kramer, W. and Fritz, H. J., Methods in Enzymology, 154, 350 (1987); Zoller, M. J. and Smith, M. Methods in Enzymology, 100, 468 (1983)) can in particular be mentioned. Modified DNA synthesis can be carried out as indicated above and in particular by using well known chemical synthesis techniques such as for example the phosphotriester method [Letsinger, R. L and Ogilvie, K. K., K. Am. CHEM. Soc., 91, 3350 (1969); Merrifield, R. B., Sciences, 150, 178 (1968)] or the phosphoamidite method [Beaucage, S. L and Caruthers, M. H., Tetrahedron Lett., 22, 1859 (1981); McBRIDE, L. J. and Caruthers, M. H. Tetrahedron Lett., 24 245 (1983)] or also the combination of these methods.

The polypeptides of the present invention can therefore be prepared using techniques known to a person skilled in the art, in particular partially by chemical synthesis or also by the recombinant DNA technique by expression in a procaryotic or eucaryotic host cell as indicated hereafter.

A particular subject of the present invention is the process four the preparation of the recombinant protein CATFIIIA having the amino acid sequence SEQ ID No 3 comprising the expression of the DNA sequence as defined above in an appropriate host then isolation and purification of the said recombinant protein.

To produce the polypeptide of the present invention, recombinant DNA techniques using genetic engineering and cell culture methods known to a person skilled in the art can in particular be used. The following stages can then be carried out: firstly preparation of the appropriate gene, then incorporation of this gene into a vector, transfer of the carrier vector of the gene into an appropriate host cell, production of the polypeptide by expression of the gene, isolation of the polypeptide, the polypeptide thus produced can then be purified.

The polypeptides of the present invention obtained by expression of the polynucleotides of the present invention can be purified from cell cultures transformed by methods well known to a person skilled in the art such as precipitation with the ammonium sulphate or ethanol, extraction under acid conditions, anion or cation exchange chromatography, hydrophobic interaction chromatography affinity chromatography, hydroxylapatite chromatography and high performance liquid chromatography (HPLC). Techniques well known to a person skilled in the art can be used to regenerate the protein when it is denatured during its isolation or purification.

The DNA sequences according to the present invention and in particular SEQ ID No1 and SEQ ID No 2 can be prepared according to techniques known to a person skilled in the art in particular by chemical synthesis or by screening of a gene bank or a cDNA bank using synthetic oligonucleotide probes by known hybridization techniques, thus amplification of DNA from isolated fragments or also by reverse transcriptase from messenger RNA (mRNA). The advantage of the technique comprising firstly the isolation of mRNA by extraction of the total RNA then the synthesis of cDNA from these mRNA by reverse transcriptase in particular rests on the fact that the mRNA do not contain introns even though these non-coding sequences are presented in the genomic DNA.

The usual cloning techniques known to a person skilled in the art and in particular described in the book by Sambrook, J. Fritsh, E. F. § Maniatis, T. (1989) entitled: ‘Molecular cloning: a laboratory manual, Laboratory, Cold Spring Harbor N.Y. can then be carried out. In these techniques, cloning can be carried out by insertion of a fragment into a plasmid which can be provided with a suitable commercial kit then transformation of a bacterial strain by the plasmid thus obtained. In particular the XL1 Blue or DH5 alpha E. coli strain can be used. The clones can then be cultured in order to extract the plasmid DNA according to standard techniques known to a person skilled in the art referred to above (Sambrook, Fritsh and Maniatis). The DNA sequencing of the amplified fragment contained in the plasmid DNA can then be carried out.

The polypeptides of the present invention can be obtained by expression in a host cell containing a polynucleotide according to the present invention and in particular a DNA sequence coding for a polypeptide of the present invention preceded by a suitable promoter sequence. The host cell can be a procaryotic cell, for example E. coli or a eucaryotic cell such as yeast such as for example ascomycetes amongst which is saccharomyces or also mammalian cells such as Cos cells for example.

A particular subject of the present invention is the expression vector containing a DNA sequence as defined above. In the expression vector, such a DNA sequence is therefore in particular the DNA sequence of the CAtfIIIA gene coding for a protein with the biological function of the transcription factor CATFIIIA of Candida albicans containing the nucleotide sequence SEQ ID No1.

In the expression vector, such a DNA sequence is thus more particularly the DNA sequence starting with nucleotide 720 and finishing at nucleotide 1955 of SEQ ID No 1.

In the expression vector, such a DNA sequence is thus also more particularly that of the CAtfIIIA gene as defined above coding for the amino acid sequence SEQ ID NO3.

In the expression vector, such a DNA sequence is thus a DNA sequence as defined above coding for the transcription factor CATFIIIA as well as the DNA sequences which hybridize with it and/or have a significant homology with this sequence or fragments of it, or also DNA sequences comprising modifications introduced by suppression, insertion and/or substitution of at least one nucleotide coding for a protein having the same biological activity as the transcription factor CATFIIIA.

In the expression vector, such a DNA sequence is in particular a DNA sequence as defined above as well as similar DNA sequences which have a nucleotide sequence homology of at least 50% or at least 60% and preferably at least 70% with the said DNA sequence or also similar DNA sequences which code for a protein, the AA sequence of which has a homology of at least 40% and in particular of 45% or of at least 50%, rather at least 60% and preferably at least 70% with the AA sequence coded by the said DNA sequence.

The expression vectors are vectors allowing the expression of the protein under the control of a suitable promoter. Such a vector can be a plasmid, a cosmid or viral DNA. For the procaryotic cells, the promoter can for example be the lac promoter, the trp promoter, the tac promoter, the β-lactamase promoter or the PL promoter. For the yeast cells, the promoter can be for example the PGK promoter or the GAL promoter. For mammalian cells, the promoter can for example be the SV40 promoter or adenovirus promoters.

Baculovirus type vectors can be also used for the expression in insect cells.

The host cells are for example procaryotic cells or eucaryotic cells. The procaryotic cells are for example E. coli, Bacillus or Streptomyces. The eucaryotic host cells include yeasts as well as of the cells of higher organisms, for example mammalian cells or insect cells. The mammalian cells are for example fibroblasts such as hamster CHO or BHK cells and monkey Cos cells. The insect cells are for example SF9 cells.

The present invention therefore relates to a process which comprises the expression of a polynucleotide according to the present invention coding for the CATFIIIA protein in a host cell transformed by a polynucleotide according to the present invention and in particular a DNA sequence coding for the amino acid sequence SEQ ID No 3. In the implementation of such a process, the host cell is in particular a eucaryotic cell.

For the implementation of the present invention, the vectors used can for example be pGEX or pBAD and the host cell can be E. coli or for example the vector pYX222 and the host cell can be in particular Saccharomyces cerevisiae.

A particular subject of the present invention is the host cell transformed with a vector as defined above and containing a DNA sequence according to the present invention.

A subject of the present invention is therefore the process for the preparation of a recombinant protein according to the present invention, as defined above, in which the host cell is DH5 alpha E. coli or XL1-Blue E. coli or in particular Saccharomyces cerevisiae.

A detailed account of the conditions under which the operations indicated above can be carried out is given hereafter in the experimental part. A plasmid is thus obtained in which the gene of the present invention is inserted and this plasmid introduced into a host cell is then obtained by operating according to the usual techniques known to a person skilled in the art.

A very precise subject of the present invention is the plasmid deposited at the CNCM under the number I-2072.

It therefore particularly relates to the XL1-Blue/Yep24-Catfc2 strain containing the CAtfIIIA gene according to the present invention.

This gene corresponds therefore to the sequence 720-1955 of SEQ ID No1.

The operating conditions under which the present invention was carried out are described hereafter in the experimental part.

The TFIIIA protein coded by the CAtfIIIA gene is therefore a transcription factor. In fact, the TFIIIA protein coded by the gene of the present invention has a biological role as a protein binding to the DNA and would be useful as transcription factor.

In particular, the gene of the present invention is expressed in different tissues and plays an important role in the initiation of the transcription of the 5s ribosomal RNA gene. The study of these factors can also be useful in the analysis of transcription regulation mechanisms.

A subject of the present invention is therefore a process for screening antifungal products characterized in that it comprises a stage where the activity of transcription factor CATFIIIA as defined above is measured in the presence of each of the products whose antifungal properties need to be determined and the products with an inhibitory effect on this activity are selected.

The demonstration within the scope of the present invention of the functional homology of the transcription factors of Candida albicans and Saccharomyces cerevisiae, illustrated in the experimental part hereafter, make it possible to envisage numerous applications for the transcription factor CATFIIIA of the present invention.

In particular because of the fact that it appears that the activity of SCTFIIIA is essential for cell survival, substances which inhibit this activity can be used as antifungal agents, either as medicaments or at an industrial level.

For example, to screen antifungal substances such as substances active on Candida albicans, the activity of CATFIIIA or one of its functional homologues constituted by a TFIIIA transcription factor is measured in the presence of each of the products whose antifungal properties need to be determined and the products having an inhibitory effect on this activity are selected.

Such screening can be carried out by measuring the transcription activity of TFIIIA in the presence of activators or of potential inhibitors to be tested. The transcription of 5S RNA can for example be measured in vitro directly by detecting the synthesis of the 5S RNA in an appropriate reaction medium.

The transcription activity can also be measured in vivo by a cell viability test. For example, transcription activity can be favourably measured in mutant Saccharomyces cerevisiae cells not expressing SC TFIIIA transformed by the CAtfIIIA gene.

The invention also encompasses the use of a product selected as indicated above for its properties of inhibiting a TFIIIA transcription factor in order to obtain of an antifungal agent.

The present invention will be better understood by reference to the experimental part which follows and which describes the cloning of the CAtfIIIA gene of the present invention.

A subject of the present invention is thus the use of a product selected by the process of screening antifungal products as defined above in order to obtain an antifungal agent.

A subject of the present invention is also the use of the transcription factor CAtfIIIA gene of Candida albicans or the transcription factor coded by this gene as defined above for the selection of a product having antifungal properties as defined above and used as inhibitor of the transcription factor of Candida albicans.

A subject of the present invention is also pharmaceutical compositions containing at least one inhibitor of the transcription factor of Candida albicans as defined above as active ingredient.

Such compositions can in particular be useful for treating topical and systemic fungal infections.

The pharmaceutical compositions indicated above can be administered by oral, rectal, parenteral route or by local route as a topical application on the skin and mucous membranes or by injection, by intravenous or intramuscular route. These compositions can be solid or liquid and be presented in all the pharmaceutical forms currently used in human medicine such as, for example, plain or sugar coated tablets, gelatin capsules, granules, suppositories, injectable preparations, ointments, creams, gels and aerosol preparations; they are prepared according to usual methods. The active Ingredient can be incorporated in excipients normally used in these pharmaceutical compositions, such as talc, gum arabic, lactose, starch, magnesium stearate, the cocoa butter, aqueous or non aqueous vehicles, fatty substances of animal or vegetable origin, paraffin derivatives, glycols, various wetting, dispersing or emulsifying agents, and preservatives.

The dose will be variable according to the product used, the subject treated and the disease in question.

A particular subject of the present invention is thus the use of compositions as defined above such as antifungal agents.

A subject of the present invention is also a method of inducing an immunological response in a mammal comprising the inoculation of this mammal with the polypeptide according to the present invention as defined above or a fragment of this polypeptide having the same function in order to produce an antibody protecting the animal against the disease.

A subject of the present invention is therefore antibodies directed against the polypeptides of the present invention as defined above having the transcription factor function CATFIIIA or against a fragment of these polypeptides having the same function and coded by the polynucleotides of the present invention and in particular by a DNA sequence as defined above.

The polypeptides of the present invention can thus be used as immunogens to produce immunospecific antibodies of these polypeptides. The term antibody designates antibodies which can equally be monoclonal, polyclonal, chimeric, single chain, non-human antibodies and human antibodies, as well as Fab fragments, including the products of a Fab immunoglobulin bank. The antibodies produced against the polypeptides of the present invention can be obtained by administration of the polypeptides of the present invention or fragments carrying epitopes, their analogues or also animal cells, preferably non-human, by using routine protocols for the preparation of monoclonal antibodies. Such antibodies can be prepared by methods well known in this field such as those described in the book Antibodies, Laboratory manual Ed. Harbow and David Larre, Cold Spring Harbor laboratory Eds, 1988.

A very particular subject of the present invention is thus an antibody directed against the CATFIIIA protein of the present invention or a fragment of this protein in particular having the same function.

A subject of the present invention is also the use of the CAtfIIIA transcription factor gene or the transcription factor coded by this gene as defined above for the preparation of compositions which can be used for the diagnosis or treatment of diseases caused by the pathogenic yeast Candida albicans.

The present invention also relates to the use of the polynucleotides of the present invention as diagnostic reagents. The detection of a polynucleotide according to the present invention coding for the TFIIIA protein of Candida albicans or of its analogues in a eucaryotic cell in particular a mammalian cell and more particularly a human being, can constitute a means of diagnosing a disease: thus, such a polynucleotide according to the present invention and in particular a DNA sequence can be detected by a wide variety of techniques in a eucaryotic cell in particular a mammal and more particularly a human being, infected by an organism containing at least one of the polynucleotides of the present invention. The nucleic acids for such a use as a diagnostic tool can be detected in infected cells or tissues, such as bone, blood, muscle, cartilage or skin. For this detection, the genomic DNA can be used directly or also be amplified by PCR or another amplification technique. The RNA or DNA and cDNA can also be used with the same purpose. By amplification techniques, the line of the mycete present in a eucaryote in particular a mammal and more particularly a human being, can be characterized by analysis of the genotype. Deletions or insertions can be detected by a change in the size of the amplified product in comparison with the genotype of the reference sequence. The points of mutation can be identified by hybridization of the DNA amplified with the sequences, labelled by a radioactive element, of polynucleotides of the present invention. Perfectly complementary sequences can therefore be distinguished from the duplex which poorly resist digestion by nucleases. The DNA sequence differences can also be detected by alterations in the electrophoretic mobility of DNA fragments in gels, with or without denaturing agent, or by direct DNA sequencing (reference: Myers et al. Science, 230: 1242 (1985)). Sequence changes at specific locations can also be revealed by protection experiments against nucleases such as RNase I and S1 or by chemical cleavage methods (reference: Cotton et al., Proc Natl Acad Sci, USA, 85: 4397-4401 (1985). Cells containing one of the polynucleotides of the present invention carrying mutations or polymorphisms can also be detected by a large number of techniques making it possible in particular to determine the serotype. For example, the RT-PCR technique can be used to detect the mutations. It is particularly preferable to use RT-PCR techniques in conjunction with automatic detection systems, such as for example the GeneScan technique. RNA and cDNA can be used in the PCR or RT-PCR techniques. For example, complementary primers of polynucleotides coding for the polypeptides of the present invention can be used to identify and analyse the mutations.

Primers can therefore be used to amplify an isolated DNA from the infected individual. In this way mutations in the DNA sequence can be detected and used to diagnose the infection and determine the serotype or the classification of the infectious agent. Such techniques are standard for a person skilled in the art and are described in particular in the manual ‘Current Protocols in Molecular Biology’, Ausubel et al, ed. John Wiley § sons, Inc., 1995).

The present invention therefore relates to a process of diagnosing a disease and preferably a fungal infection caused in particular by Candida albicans such as mycoses as indicated above, this process comprising the determination from a sample taken from an infected individual, an increase in the quantity of polynucleotide of the present invention. Such a polynucleotide can in particular have a DNA sequence of the present invention as defined above.

Increases or reductions in the quantity of polynucleotides can be measured by techniques well known to a person skilled in the art such as in particular amplification, PCR, RT PCR, Northern blotting or other hybridization techniques. In addition, a diagnosis method in accordance with the present invention consists of the detection of too large an expression of polypeptides of the present invention, in comparison with control samples constitued by normal, non-infected tissues used to detect the presence of an infection. The techniques which can therefore be used to detect the quantities of proteins expressed in a host cell sample are well known to a person skilled in the art. For example the radioimmunoassay or competitive-binding techniques, Western Blot analysis and ELISA test (ref Ausubel indicated above) can thus be mentioned.

A subject of the present invention is also a kit for the diagnosis of fungal infections comprising a DNA sequence according to the present invention as defined above or a sequence having a similar function or a functional fragment of this sequence, the polypeptide coded by this sequence or a polypeptide fragment having the same function or an antibody directed against such a polypeptide coded by this DNA sequence or against a fragment of this polypeptide.

This kit can thus contain a DNA sequence according to the present invention as defined above and for example the DNA sequence SEQ ID No1 or a fragment of this sequence or also the sequence 720 to 1955 of SEQ ID No1.

Such kit could also contain a polypeptide according to the present invention or a fragment of this polypeptide and in particular the protein having the AA sequence SEQ ID No3 or also an antibody as defined above.

Such a kit can be prepared according to methods well known to a person skilled in the art.

The sequences SEQ ID No 1 to 9 indicated in the present invention are described hereafter.

The experimental part hereafter makes it possible to describe the present invention without however limiting it.

Experimental Part

### EXAMPLE 1

### Cloning and Sequencing of the CAtfIIIA Gene

a) Culture Conditions:

The bacteria Escherichia coli (E. coli) of the DH5 alpha (Gibco BRL) or XL1-Blue type K12 (Stratagene) line was used for the preparation of the plasmids of the present invention. The growth of this bacteria was carried out according to usual conditions in liquid LB medium which contains 10 g of bactotryptone, 5 g of yeast extract and 10 g of NaCl per litre of water and which also contains 100 micro g/ml of ampicillin (SIGMA).

The colony was removed onto solid LB+agar+ampicillin medium then cultivated in 100 ml of LB medium and incubated to OD (600 nm)=0.8.

The incubation was carried out at 37° C. under a normal atmosphere and agitation at 225 rpm.

The viability of the strain is verified when the strain grows on LB+ampicillin medium at 100 micro g/ml.

It can be noted that a gene resistant to the Bla ampicillin forms part of the vector in which the fragments of CAtfIIIA are cloned. Therefore, the selection of strains containing the plasmids containing the tfIIIA gene of Candida albicans of the present invention can be carried out by culture of the strains in this medium containing ampicillin (100 micro g/ml), such a medium only allowing the survival of strains which contain the gene resistant to the ampicillin and therefore only strains which contain the tfIIIA gene of Candida a. of the present invention.

For the preservation of the strains obtained, 15% of glycerol is added to the culture medium: the cultures are therefore preserved in the suspension medium LB+100 micrograms/ml of ampicillin+15% of glycerol at the bacterial concentration of OD (600 nm=0.8 in the form of aliquots in cryotubes of 1 ml per tube.

For the sequencing, the plasmid DNA of several bacteria originating from each of the cloning operations indicated hereafter is prepared using a commercial kit (Qiagen Plasmids kit). The fragments corresponding to the sequence of the CAtfIIIA gene are sequenced on the two strands according to standard techniques known to a person skilled in the art (use of the ABI 377 XL sequencer, Perkin Elmer).

b) Cloning and Sequencing of the CAtfIIIA Gene:

Within the scope of the present invention, the gene coding for the transcription factor CA i.e. SEQ ID No1 represented in FIG. 1 was isolated from the gene fragment bank of Candida albicans. (Sanglard et al., Antimicrobial agents and chemotherapy 39, 2378-2386, (1995)).

The structure of the gene was identified by sequencing. The strategy used rests on the hypothesis that SC and CA are similar yeasts the gene structure of which can be homologous. The following process is then carried out:

Within the scope of the present invention, by using the Standford internet site which makes it possible to access the preliminary sequences of the Candida albicans genome, a fraction of sequence homologous with S. cerevisiae tfIIIA was identified. This fragment contains an open reading frame (258 bp) coding for a protein for which two zinc finger moieties and a region rich in serine residues characteristic of the TFIIIA factor of SC can be identified. This open reading frame in reality contains 259 nucleotides. In order to amplify the fragment corresponding to Candida albicans, two oligonucleotides were selected from this sequence. These oligonucleotides are the following:
INT CAND located in the position 720-740 of SEQ-ID No1 and called SEQ ID No4 and
3′ CAND located in the position 955-978 of SEQ ID No1 and called SEQ ID No5.

A fragment of 259 base pairs is thus obtained.

It was firstly confirmed by PCR that it is possible to amplify a fragment of CA genomic DNA, prepared from CA cells by the usual methods known to a person skilled in the art, and on the other hand in the CA gene bank. These oligonucleotides have also made it possible to synthesize a fragment of DNA from genomic DNA of Candida albicans in order to prepare a probe labelled with 32P (phosphorus 32) using a kit (Mega Prime, Amersham).

This fragment was used for the screening of the bank of genomic Sau 3A Fragments of Candida albicans cloned in the BamHI site of the vector YEp24 (multicopy-Ura3) [Botstein et al., Gene, 8, 17-24, (1979)].

The DH5 alpha E. coli cells transformed with the vector YEp24 (multicopy vector with selection gene URA3) containing the fragments described above (17000 clones) are plated on dishes containing a LB+ampicillin medium and cultured at 37° C.

A replica on nitrocellulose filter is then treated by techniques known to a person skilled in the art such as for example NaOH: 0.5M, 5 minutes; Tris-HCl: 1M (pH=7.5) 5 minutes; NaCl 1.5M/Tris-HCl 0.5M (pH 7.5).

As regards drying, the filters are kept for 10 minutes at 80° C. then fixed with UV (Stratalinker). Pre-hybridization and hybridization are carried out in a NaPO4 buffer (pH 7.2) 0.5M; EDTA 10 mM; SDS 7% (ref., Church and Gilbert, PNAS 81: 1991 (1984)).

The probe is labelled with 32P with the MegaPrime and (alpha 32P) dCTP kit (Amersham UK). The hybridization is carried out overnight at 65° C. The filters are then washed in 1% SDS, 40 mM NaPO4 (pH 7.2), six times for 5 minutes at 65° C. and they are then subjected to autoradiography overnight. Hybridization on a filter with the probe labelled with 32P has made it possible to select several positive clones which have been recultured in dishes in order to isolate them.

Individual clones have thus been isolated.

Three types of clones are thus obtained which are called 9, 18 and 47 containing three different inserts of the CAtfIIIA gene of the present invention: analysis by PCR confirmed the presence of the 259 bp fragment.

The YEp24 plasmids containing Candida albicans inserts were collected from these colonies. The restriction map of each of these plasmids was established and made it possible to note that all the inserts originate from the same region of the Candida albicans genome. For the sequencing of this region the following oligonucleotides were used:
INT-Cand located at position: 720-740 of SEQ ID No 1 and called SEQ ID No 4
3′-Cand located at position: 955-978 of SEQ ID No 1 and called SEQ ID No 5
Cont-Int located at position: 719-741 of SEQ ID No 1 and called SEQ ID No6
Can-Kor1 located at position 1365-1389 of SEQ ID No 1 and called SEQ ID No7
   and the sequencer ABI 377 XL (Perkin Elmer). The sequencing of this region made it possible bring the following points to light:
1) The three clones all contain only one open reading frame, uninterrupted for 1236 bp with the same sequence which codes for a protein.
2) The open reading frame codes for a 412 AA protein which shows a significant homology with the TFIIIA factor of Saccharomyces cerevisiae. Analysis of the protein makes it possible to find the 9 zinc finger moieties which are characteristic of the transcription factor TFIIIA. Comparison of the proteinic sequences of SC CATFIIIA and TFIIIA, makes it possible to demonstrate a similarity of 50% and an identity of 45%. For the amino acid translation the fact that in Candida albicans the CTG codon is translated to serine and that there are 2 CTG codons in Candida albicans TFIIIA was taken into account.

The following should be noted:
The preservation of the Serine rich region in the N-terminal part.
the presence of a very long intermediate region between the 8 and 9 zinc fingers characteristic of SC.

The sequence differences between the TFIIIA proteins of SC and TFIIIA of Candida albicans is located in the C-terminal part outside the zinc finger moieties.

The YEp24 plasmid containing the promoter region and the sequence coding for CATFIII was transformed in the XL1 Blue E. Coli strain then deposited under the number I-2072 at the CNCM, Institut Pasteur 2,5 rue of Docteur ROUX 75015 Paris, on the 15 Sep. 1998.

### EXAMPLE 2

### Expression of the tfIIIA Gene

A fragment contained in clone 9 was amplified by PCR using primers containing sequences recognized by the restriction enzymes EcoRI and XhoI and hybridizing with the tfC2 gene, the primers are the following:
5-EcoTF located at position 720-732 of SEQ ID No1 and called SEQ ID No8 and
3′-XhoI located at position 1946-1960 of SEQ ID No1 and called SEQ ID No9.

Amplification by PCR of the genomic DNA is then carried out in the following manner:
0.5 micrograms of DNA of clone 9 is added to 50 microlitres of a reaction solution containing 200 nanograms/ml of each dNTP, the primers indicated above at a rate of 25 micromoles/l for each, 2 mM MgCl2, 1× Pfu Buffer, 5U Pfu polymerase (Perkin Elmer).

The reaction medium is subjected to 30 PCR cycles each corresponding to 94° C. for 30 seconds, then 60° C. for 45 seconds then 72° C. for 1 minute.

The fragment containing the coding sequence for CATFIII was sub-cloned in the vectors pYX122 (CEN, HIS 35 and pYX222 (2 micron, HIS3) (R and D System). This plasmid was used to transform Saccharomyces c cells. YWRI (Mat alpha, can 1-100, his 3-11, leu 2-3, 112 trp 1-1, ura 3-1; ade 2-1, tfC2: leu2+pJA230), (Camier and al, Proc. Natl. Acad. Sci. 92 9338-9342, 1995).

The strain transformed according to the same methods as those indicated above allows the expression of the transcription factor TFIIIA of Candida albicans containing a HA tag.

Conclusion

The experimental implementations indicated above therefore show the following points:
1) The TFIIIA factor gene of Candida albicans was isolated in three clones 9, 18 and 47 obtained as indicated above in Example 1 from the gene bank of Candida albicans using a hybridization technique. The structure of this gene was identified by sequencing.
2) The CATFIIIA protein of the CAtfIIIA gene obtained in Example 1 is constituted by 412 AA and shows a high homology with the SC TtIIIA factor. This protein contains a region rich in SER residues in the N-terminal and 9 zinc finger part, the arrangement of which is identical to that of the TFIIIA-protein of SC.
3) The sub-cloning of the gene of the TFIIIA factor of Candida albicans was carried out and the gene was placed under the control of an SC promoter.

1-11. (canceled)

12. A polypeptide having the transcription factor function CATFIIIA and having the amino acid sequence SEQ ID No: 3 coded by the DNA sequence of the CAtfIIIA gene coding for a protein having the biological function of transcription factor of candida albicans CATFIIIA containing the nucleotide sequence of SEQ ID No: 1 and the analogues of this polypeptide.

13-23. (canceled)

24. A method of inducing an immunological response in a mammal comprising the inoculation of the mammal with the polypeptide as defined in claim 12 or a fragment of this polypeptide having the same function to produce an antibody making it possible to protect the animal against the disease.

25-26. (canceled)

27. Kit for the diagnosis of fungal infections comprising a DNA sequence as defined in claim 5 or a functional fragment of this sequence, the polypeptide coded by this sequence or a polypeptide fragment having the same function or an antibody directed against such a polypeptide coded by this DNA sequence or against a fragment of this polypeptide.
